(19) United States
(12) Patent Application Publication
(10) Pub. No.: US 2020/0390375 A1
(43) Pub. Date: Dec. 17, 2020

(54) PERIPHERAL FIBEROPTIC INTRAVASCULAR BLOOD METRIC PROBE MODULAR DEVICE AND METHOD

(71) Applicant: PULSION MEDICAL SYSTEMS SE, FELDKIRCHEN (DE)

(72) Inventor: Robert John ANDERSON, North Liberty, IA (US)

(73) Assignee: PULSION MEDICAL SYSTEMS SE, FELDKIRCHEN (DE)

(21) Appl. No.: 17/005,855

(22) Filed: Aug. 28, 2020

Related U.S. Application Data
(62) Division of application No. 15/404,902, filed on Jan. 12, 2017.
(60) Provisional application No. 62/277,724, filed on Jan. 12, 2016.

(51) Int. Cl. A61B 5/1459; A61B 5/0215; A61B 5/145; A61B 5/1455; A61M 25/00

(52) U.S. Cl. CPC A61B 5/1459; A61B 5/0215; A61B 5/14539; A61B 5/14552; A61M 39/105; A61M 25/003; A61B 2562/0233; A61B 2562/223; A61B 5/14556

(57) ABSTRACT

The invention is a device and method for measuring, inter alia, blood oxygenation, arterial/venous blood gas, and/or hemoglobin values in an already-inserted arterial or venous catheter in a patient. It allows the measurement of a meaningful and commonly understood metrics of blood oxygenation and gas exchange in hypo-perfused patients while avoiding the additional discomfort and risk of infection posed by inserting a standalone device with its own catheter. Analogous uses of the apparatus for other measurements is possible.

### CROSS-REFERENCE TO RELATED APPLICATION

This application claims the benefit of Provisional Application U.S. Ser. No. 62/277,724 filed on Jan. 12, 2016, all of which is herein incorporated by reference in its entirety.

### BACKGROUND OF THE INVENTION

Oxygen saturation is the measure of oxygen attached to hemoglobin and carried in the blood; this is otherwise referred to as oxyhemoglobin saturation. Oxygen saturation can be used by medical professionals to evaluate a patient's oxygenation status and determine a patient's need for the administration of oxygen as a medical intervention. The oxygen saturation value is reported as a percentage (SpO2) of the maximum amount of oxygen that the blood can bind. A healthy individual will have an arterial blood SpO2 level between 95%-100%. If the arterial SpO2 drops below 90 percent in a healthy adult, internal organs are at risk of not receiving sufficient oxygen to maintain life.

The most commonly used medical device for measuring a patient's oxyhemoglobin saturation level is a non-invasive oximetry probe placed (clamped) on an extremity of the patient; usually one of the fingers, toes, earlobes, or forehead. Such an oximeter sends two wavelengths of light from one side of the clamp through the patient's capillary beds to the opposite side. The other side of the clamp has a photo detector that reads the amount of light transmitted, which can be translated into an oxygen saturation value. Such devices can produce inaccurate readings for a variety of reasons, including hypoperfusion, callused distal extremities, or the presence of an opaque layer on the finger nail such as nail polish. Non-invasive oximetry probes also regularly fall off the patient thereby preventing continuous, reliable data. Non-invasive probes can cause pressure ulcers on the extremities or cause minor burn marks if they are not regularly repositioned. Additionally, inaccurate pulse oximetry measurements lead to increased alarms which can lead to documented healthcare provider alarm fatigue. In cases of hypoperfusion, SpO2 readings from the extremities do not match those of central SaO2 (oxygen saturation of arterial blood) levels, as they do in healthy patients. In most cases of hypoperfusion, non-invasively calculated SpO2 is not as adequately accurate or is entirely undetectable.

As previously noted, if arterial blood oxygenation drops below patient specific parameters, internal organs are at risk of damage secondary to hypoxemia. When non-invasive oximeter probes fail to compute reliable SpO2 values, healthcare professionals must rely on other metrics to attempt to determine a patient's level of oxygenation; this may include arterial blood gas monitoring. This method has large disadvantages, including unnecessary time consumption, patient blood supply depletion, patient discomfort, increased cost, and lack of real-time monitoring.

It is common for patients in intensive care units to experience hypoperfusion chronically. In this setting, the inability of non-invasive measures of SpO2 to provide accurate, real time readings is particularly problematic. Monitoring the health of the patients in intensive care units is especially critical. This problem is not effectively addressed in the art. While intravascular oximetry is known in the art, it is generally disfavored because of its invasiveness, requiring that a blood vessel be punctured and a catheter inserted. Because of this, there is a greater risk of infection, a need for additional sterilization, and patient discomfort. In addition, certain authors refer to intravascular oximetry in central venous vasculature to calculate a Central Venous Oxygen Saturation (ScvO2); this metric corresponds to the amount of oxygen that returns to the right atrium of the heart after the body's metabolism has extracted all the oxygen it requires at this time. A normal ScvO2 is approximately 65-75%. Such devices are designed to be inserted via their own needle and catheter assembly and terminate in the central venous system, a very invasive process. This author is not aware of any publication that refers to the use of intravascular oximetry to measure peripheral or central arterial oxyhemoglobin saturation (SaO2).

### SUMMARY OF THE INVENTION

In one aspect, the invention is a modular intravascular oximetry device designed to terminate within a peripheral artery, and a method for detecting blood oxygenation using such a device. The modularity allows this device to be inserted into any existing arterial catheter apart from brand and could be inserted at any time, at catheter placement, after placement, and removed at any time. The device sends and receives a light signal into the blood vessel via a fiber optic cable and detects the light reflected off the hemoglobin through another fiber optic cable. The device then translates these light signals into blood oxygen saturation values displayed on an external monitor. This oxyhemoglobin saturation value can be used as a metric of the overall oxygenation status of a patient. The invention measures arterial blood oxygenation directly instead of using capillary bed oxygenation as a proxy. This avoids any problem of variance between these two values due to hypoperfusion, a common condition in intensive care units. The invention also avoids the problem of introducing substantial additional risk of infection, as it is designed for use in conjunction with an existing arterial catheter that the patient already has inserted into a peripheral artery as part of the standard best practices in intensive care units. This limits the need for an additional invasive procedure and indwelling vascular line. Furthermore, the device itself and the catheter into which the device is inserted are firmly securable to the patient, and thus they would not be able to casually fall off and cease oxyhemoglobin transduction as is common with standard non-invasive probes. Use of this probe in a venous catheter is considered, although current understanding of peripheral venous oxyhemoglobin saturation normal values is not known in the literature. The probe can be used in analogous ways in other tubular structures and for other measurements.

One of the primary aims of this aspect of the invention is to allow the insertion of a modular fiber optic oximetry device into pre-existing catheter systems commonly used to measure other physiological states (i.e. blood pressure or BP) or introduce medicines or other fluids into the bloodstream. Pre-existing catheter systems are ubiquitous in hospital settings and peripherally terminating arterial catheters are commonly used in intensive care units, where hypoperfusion is common. In one embodiment, the use of a modular fiber optic oximetry device in conjunction with a pre-existing catheter line is achieved via the use of a Y-luer-lock connector. Any three-port connector could be considered (e.g. Y or T connector). The invention is not necessarily limited to the same. It is to be understood that the optics could pass through either Y-port channel; one channel usually should remain available for BP transducing or other functions. The diameter of the fiber optic cable in such an embodiment would be sufficiently small to prevent the occlusion of flow through the connector and the rest of the catheter system thereby not preventing or unduly dampening the ability of the arterial catheter to perform other and/or concurrent functions (e.g. to transduce blood pressure readings and draw blood for lab work as for which it was initially intended). In this aspect, the term modular is used to indicate, e.g., the device could be inserted into any existing arterial catheter apart from the catheter and could be inserted at any time; e.g. at catheter placement, after placement, etc., and removed at any time too.

Other aspects of the invention relate to use of a modular optical interrogation and feedback subsystem inside a catheter or other tubular structures for a variety of other possible uses. Examples include but are not necessarily limited to measuring a variety of arterial blood gases, direct hemoglobin measurement, and solid tissue measurements, as will be further discussed later.

### BRIEF DESCRIPTION OF THE DRAWINGS

FIG. 1 shows a lateral perspective view the Y-luer-lock connector assembly 14 of a first embodiment of the invention (the Y connector is but one exemplar of a three-way connector). A conventional intravascular catheter sub-assembly 22 (elongated cannula portion for insertion into a blood vessel and a cup-shaped proximal connector portion to snap-in or otherwise releasably connect to a Luer taper type connector like 14) is shown separated from luer-lock. The arterial catheter 22 is inserted into a peripheral blood arterial vessel and terminates peripherally. A proximal channel portion 18 connects the Y-luer-lock 14 out its back or proximal end to a conventional intravascular (IV) line, an arterial pressure transducing tube, or a similar tube (not pictured in FIG. 1, but see, e.g., FIGS. 14, 15A & B, and 16). A branch channel 16 (through the oblique-branch of the Y-shape of luer-lock 14) connects to distal channel portion 20 of the Y-luer-lock 14. A double fiber optic cable 2, such as are commercially available and known in the art (see, e.g., U.S. Pat. No. 8,521,248 B2, incorporated by reference; see its FIG. 1 as one example) passes through the branch channel 16 and into standard luer-lock arterial catheter 22. The fiber optic cable 2 may be permanently fixed to the Y-connector 14 to eliminate the ability for the fiber optic cable 2 to advance or migrate into the vessel. However, as explained later, the fiber optic module can be removably installed into the catheter for both retro-fitting existing catheters and selective use and removal. The module can be temporarily positioned, secured, or placed in the catheter by any number of techniques as opposed to permanently. A few non-limiting examples are adhesives, pins, clamps, or interference fit at or near the point of entry into the catheter or proximal from that. The end of the branch 16 of Y-connector 14 through which the fiber optic cable 2 is inserted can be sealed to avoid any loss of blood out of the vessel or entrainment of air into the vessel. The rest of the oximetry device, the optical module (not pictured in FIG. 1 but commercially available, see, e.g., equipment and components available for use with the model sold under the brand name PreSep Central Venous Oximetry Catheter, from Edwards LifeScience having principal offices at One Edwards Way, Irvine, Calif. 92614 U.S.A. (a device used to measure central venous oxygenation instead of peripheral arterial oxygenation; see also FIGS. 14 and 16), attaches to the proximal end of the double fiber optic cable 2. See FIG. 14 for additional information. The optical module connects to a compatible patient diagnostic monitoring unit. For additional details see also FIGS. 15A, 15B, and 16.

FIG. 2 shows a greatly enlarged cross sectional view (taken along line “FIG. 2-FIG. 2” of FIG. 1) of an embodiment of the double fiber optic cable 2. The fiber optic cable 2 connects to a light source and a photodiode (not pictured), which is in turn connected to a calculation and display apparatus (see FIG. 3).

FIG. 3 shows a reduced scale perspective view of how an exemplary embodiment of a system according to the invention operates with respect to the patient, and includes a broad view of related components of the functioning device. The standard catheter 22 enters the patient at one end and is connected to the luer-lock 14 (pictured in more detail in FIG. 1) at the other.

FIG. 4 shows a partial cross sectional perspective view (sectioned axially) of how an exemplary embodiment of a double optical fiber cable of this system would be incorporated in a standard Y-luer-lock connector such as 14.

FIG. 5 is a picture of a prototype of the instrument of FIG. 1.

FIGS. 6 and 7 are alternative enlarged-in-scale descriptions of a cross-section like that of FIG. 2.

FIG. 8 is a graph of test results.

FIG. 9 is a diagrammatic view of a set-up of an apparatus at least similar to FIG. 1 for measurement of a variety of arterial blood gases by using a multi-colored adjustable dye wheel at the light input to the fiber optic.

FIG. 10 is a diagrammatic view related to use of an apparatus at least similar to FIG. 1 for measurement of a variety of blood gases by using a fiber optic coated with a variety of different fluorescent dyes.

FIG. 11 is a diagrammatic illustration of a similar apparatus to FIG. 1 with a color wheel to change color of emitted light and a photoreceptor to receive and translate the return light.

FIG. 12 is a diagrammatic illustration of a similar apparatus to FIG. 1 with multiple light sources available to inject into the fiber optic (individually or in combination).

FIGS. 13A and B are diagrammatical illustrations of use of similar apparatus to that of FIG. 1 for solid tissue insertion and measurement.

FIG. 14 is a system diagram illustrating how an embodiment of the invention can be hooked up to an oximeter module.

FIGS. 15A and 15B are system diagrams illustrating how embodiments of the invention can be hooked up to an oximeter module or other external components.

FIG. 16 is a system diagram illustrating how an embodiment of the invention can be hooked up to a prior art oximeter module.

FIGS. 17A and B are illustrations of one example of optical connections to an optical module (FIG. 17A) and to the probe (FIG. 17B) such as can be used in the systems of FIGS. 14-16. Note that the probe connection should be one connecting piece with two optic strands/bundles, but are shown here separated.

FIG. 18 is a highly diagrammatical illustration of an embodiment of the invention as inserted in a patient's blood vessel.

FIG. 19 illustrates the fiber optic tip placed recessed back from tip of intravascular catheter or needle or tubular structure.

FIG. 20 illustrates the fiber optic tip placed at tip of intravascular catheter or needle or tubular structure.

### DETAILED DESCRIPTION OF THE EXEMPLARY EMBODIMENTS OF THE INVENTION

For a better understanding of the invention, several examples of some of the forms and embodiments it can take will now be described in detail. These examples are neither exclusive nor inclusive of all forms and embodiments the invention can take.

For example, several of the embodiments will be discussed in the context of a measuring an arterial blood gas (ABG) by combining a probe module that positions a fiber optic pair through an arterial catheter lumen to emit light through one fiber optic and receive reflectance of that emitted light from blood flowing in the artery so that the reflectance can be collected and processed into an electrical signal that can be analyzed to derive at least one ABG value by known techniques. However, the invention can be applied in analogous ways to other measurements. A few non-limited examples are venous measurements and solid tissue measurements.

For example, several embodiments will be discussed in the context of oximetry. A variety of commercially-available oximetry back-end systems or units are available to which the probe module of the present invention can be operatively connected. In analogous ways, the probe module of the present invention can be combined with other components to obtain other types of measurements.

For further example, several embodiments will be discussed where the probe module occupies approximately one-half the interior diameter of the catheter lumen. It is to be understood that this can vary. For example, it could be less if sufficiently small diameter but sufficiently effective fiber optics and any enclosure or binding of them are available relative the lumen inside diameter. The precise way the probe module occupies interior catheter lumen space can vary. For example, the probe module could be positioned in abutment with the catheter internal wall all along the catheter. But it does not necessarily have to be in abutment. As will be discussed, so long as the probe module leaves sufficient continuous space along the catheter lumen for an effective catheter function, the amount of space the probe module occupies and how it occupies that space can vary.

Furthermore, the lumen in which the probe module is placed can vary according to need or desire. As will be appreciated, it can differ in material, form factor, physical characteristics and otherwise as between a catheter for insertion into an artery or vein versus insertion into solid tissue.

These, and other objects, features, aspects and advantages of the present invention will become more apparent by reference to the specification and claims.

### I. Embodiment 1

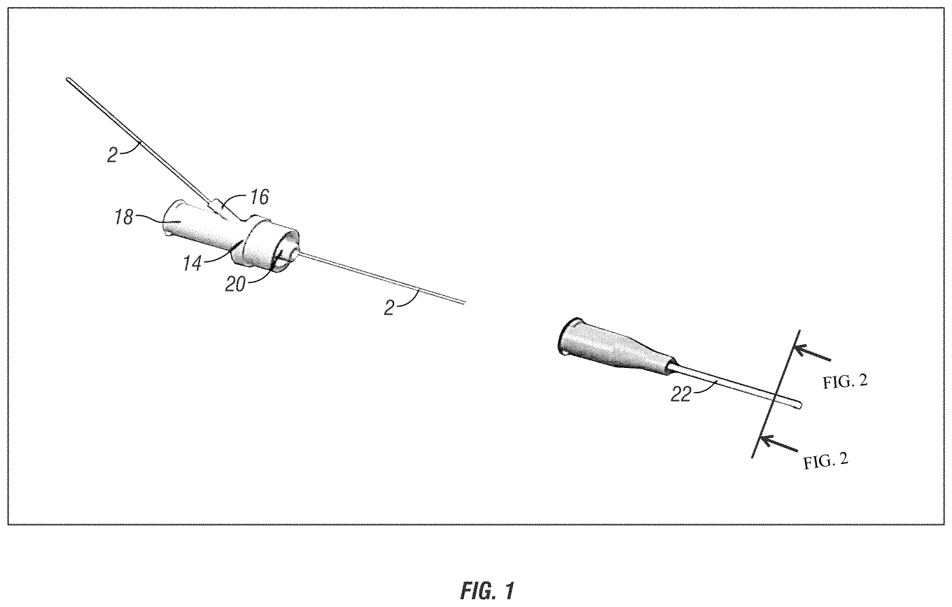

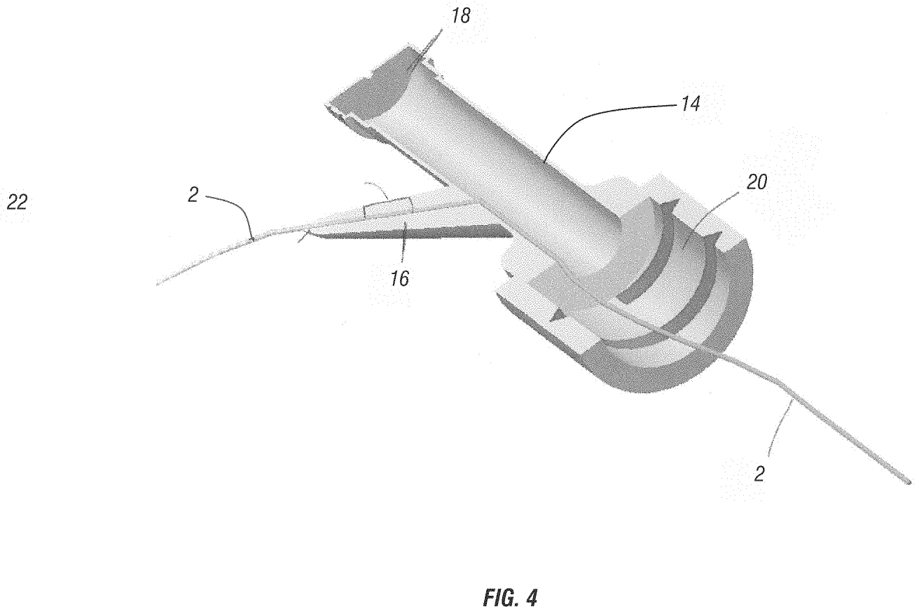

A. FIGS. 1-4

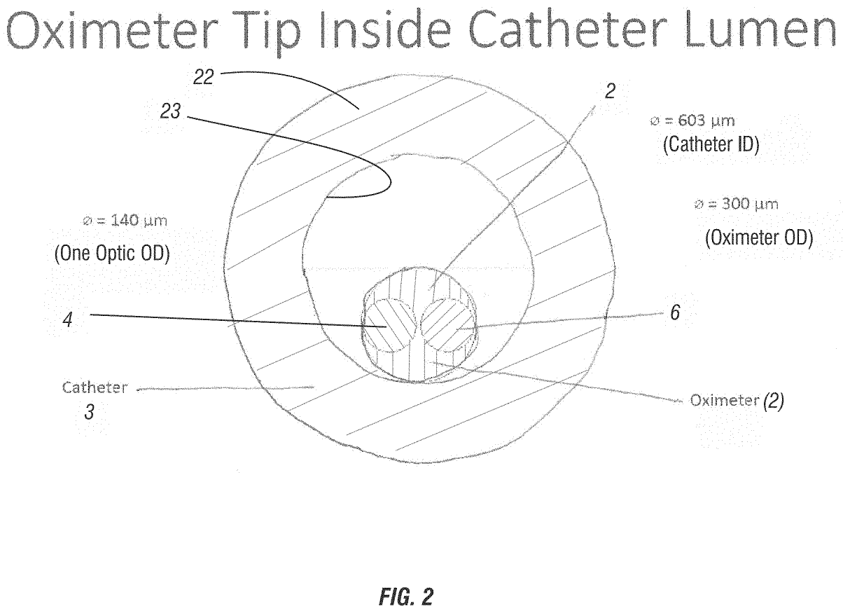

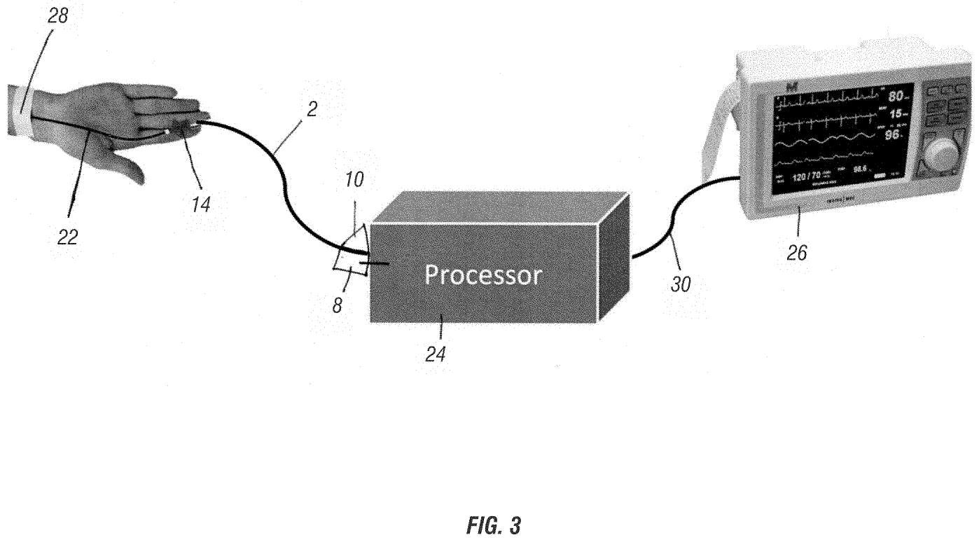

One embodiment of the invention includes a standard luer-lock arterial catheter 22, connected via a luer-lock to the distal end channel portion 20 of a Y-luer-lock connector (generally 14). Note that an intravenous catheter may be used as well based on the desired physiologic parameter to be measured (SaO2=Arterial; SvO2=Venous). A fiber optic cable (generally 2) is threaded into the arterial catheter 22 and extends at its first or distal end just beyond, or at, the distal end of the catheter 22 that terminates in a peripheral artery in the patient 28 (see at FIG. 3). Fiber optics will have standard cladding and coating as indicated by brand or size utilized per application to prevent inadvertent dissemination of emittance or receiving light signal. The fiber optic cable 2 can be fixedly attached to the interior surfaces of the Y-luer-lock 14 where the fiber optic cable 2 passes through it, entering at the Y-channel or branch channel 16, and exiting at the distal end channel portion 20. The proximal Y-channel portion 18 of the Y-luer-lock (generally 14) may be attached to an IV line, an arterial pressure transducing tube, or another similar tubing system (not shown but well known in the art). Even though the internal components of the fiber optic cable 2 are surrounded by a protective outer jacket 3 (FIG. 2), the outside diameter of the fiber optic cable 2 is small enough that it does not significantly block flow through channel portions 18 and 20 of the Y-luer-lock 14 in general or particularly the flow through distal channel portion 20. In one embodiment, by this it is meant that the outer diameter or perimeter of the fiberoptic probe is not to exceed approximately one-half the inner diameter of the intravascular catheter or open channel on the y-connector. The fiber optic cable 2 itself includes, inside jacket 3, a first fiber optic 4 attached at the fiber optic cable's second or proximal end, not inside the artery, to light source 10 (FIG. 3). The fiber optic cable 2 also includes inside jacket 3 a second fiber optic 6 attached at the second or proximal end to a photo detector 8 (FIG. 3). The photo detector 8 is electrically coupled to a processor 24 which takes the electric signal from the photo detector 8 that corresponds to the intensity of the light after passing through the patient's blood reflectance off of oxyhemoglobin, and calculates the oxygenation of the blood based on a series of natural-law-based calculations. Examples are as follows:

For a general equation:

SaO2=[HbO2]/[Total Hemoglobin]

HbO2-Oxyhemoglobin=hemoglobin with oxygen molecules bound to it.

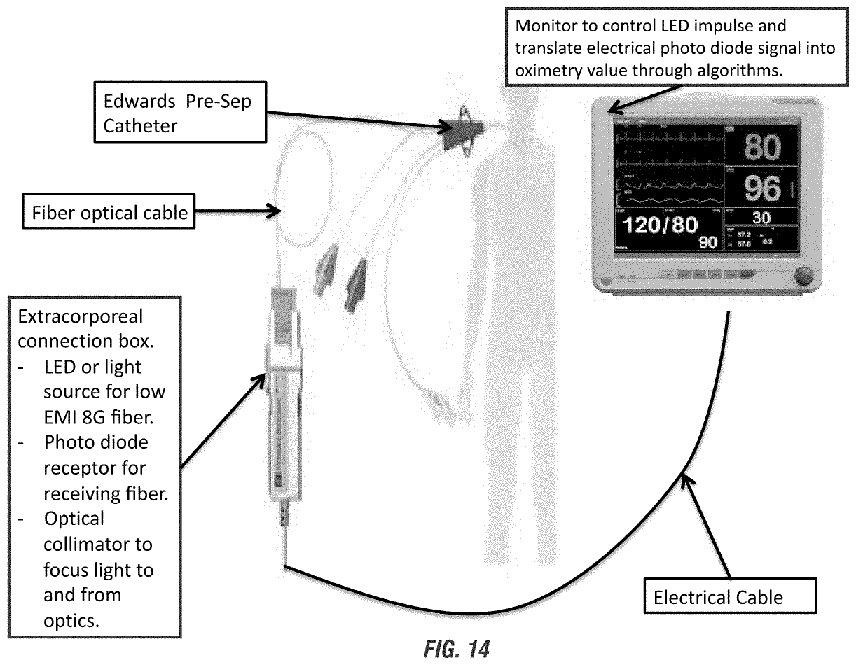

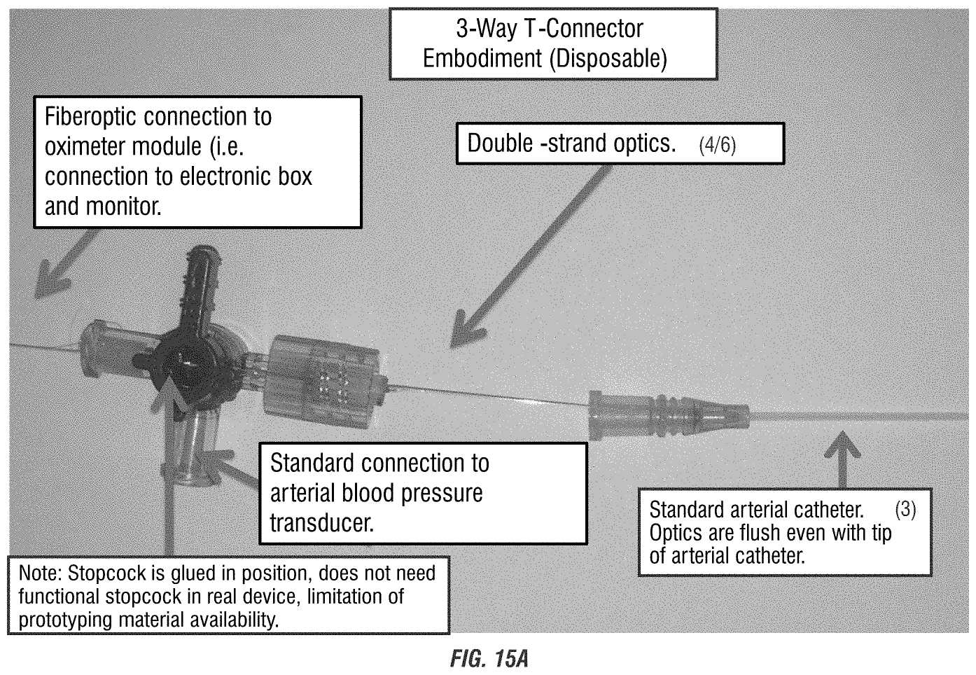

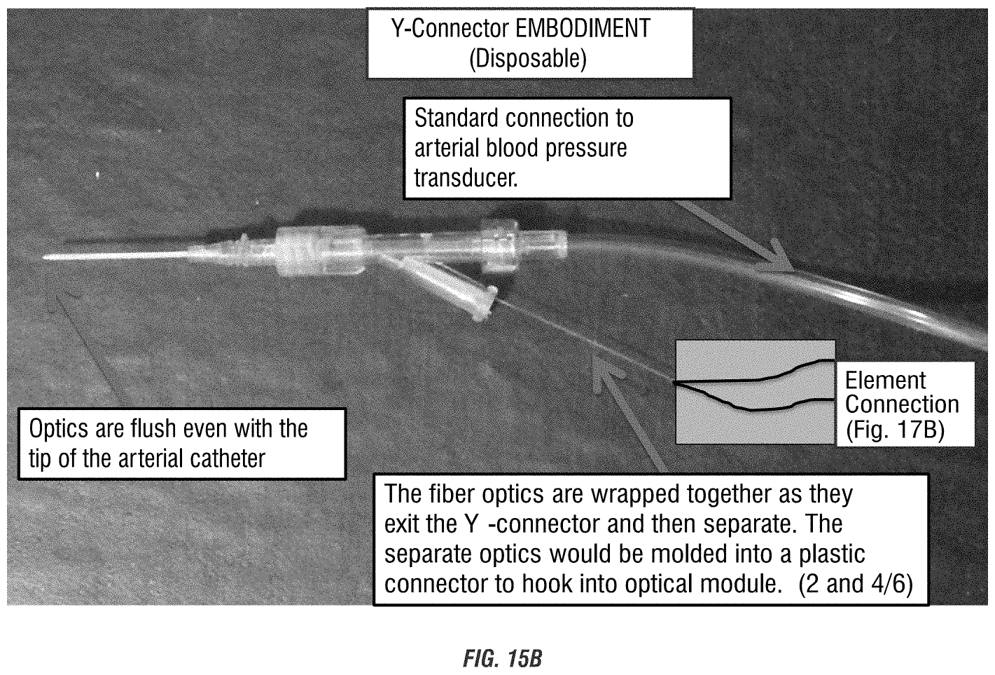

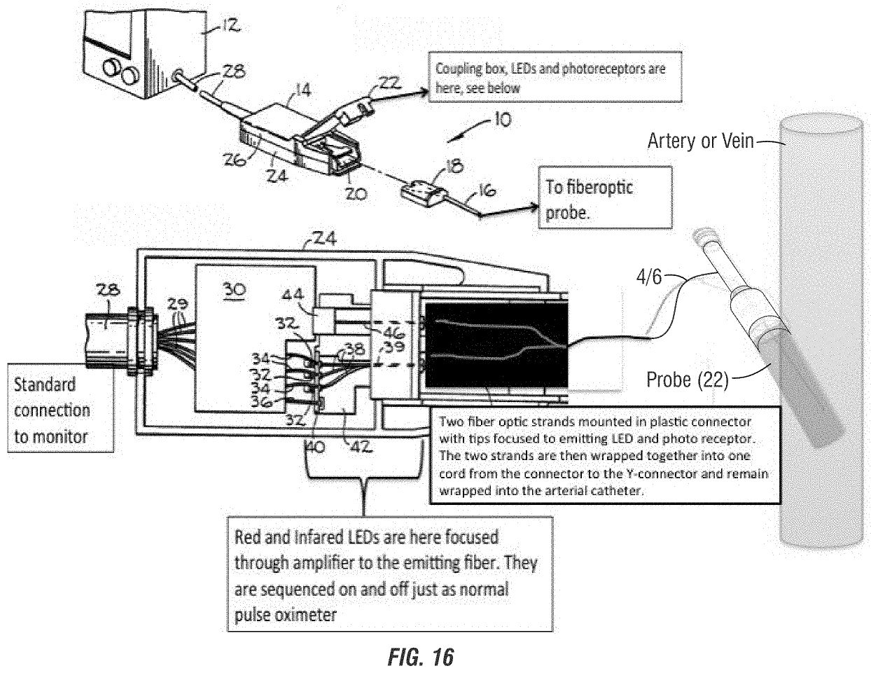

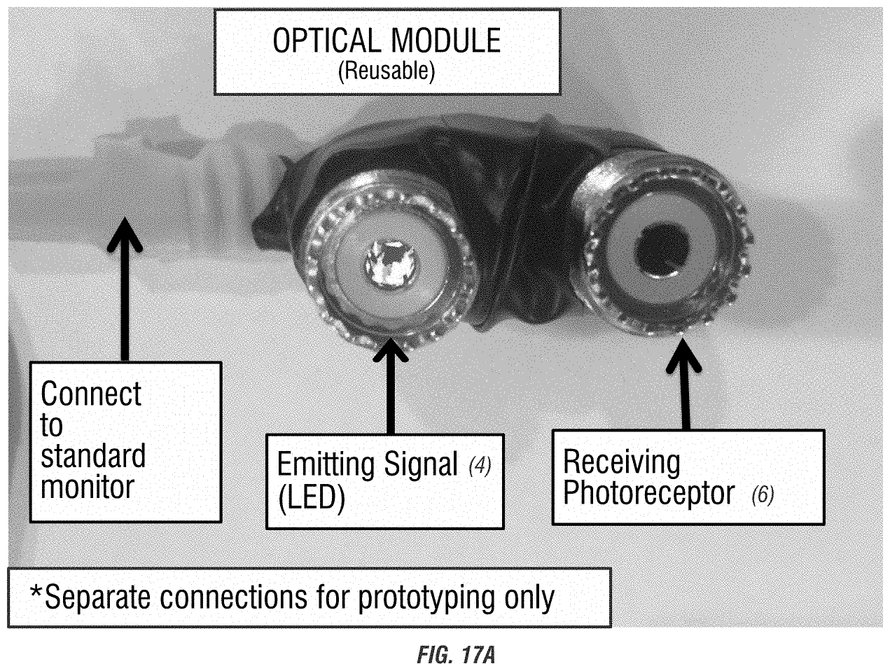

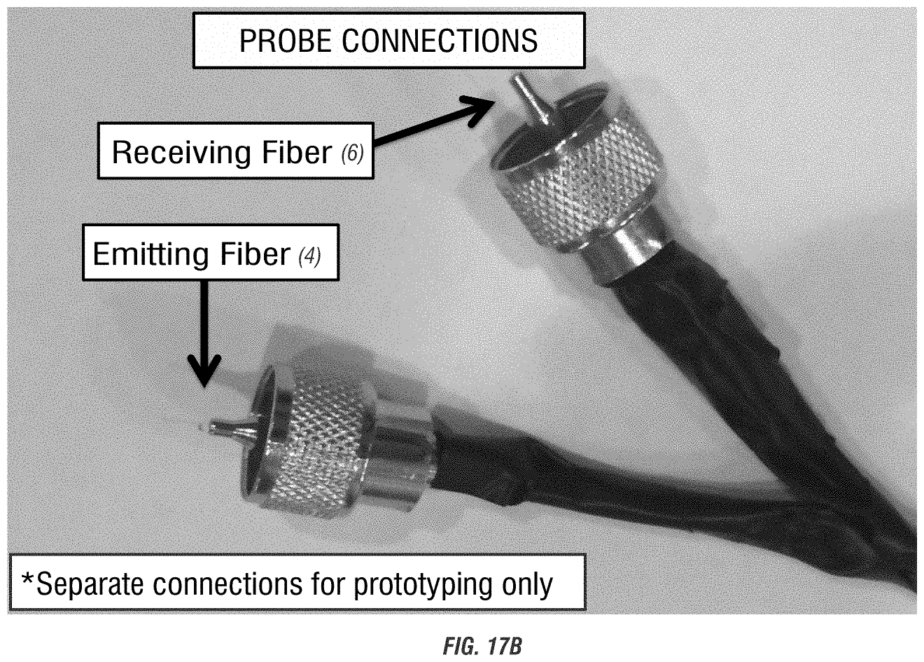

The processor 24 is connected to a display 26 via an electronic connector 30. Processor 24, display 26, double fiber optic cable 2, and luer lock assembly 14 are commercially available. Examples are Edwards EV1000 Clinical Platform (Irvine, Calif.), Phillips Healthcare Pulse Oximetry Monitoring Equipment, Teleflex Arrow Arterial Catheter (20 Ga.) at Teleflex Medical 2917 Weck Drive Research Triangle Park, N.C. 27709, Qosina Inc. Y-Connector (part 84049) at 150-Q Executive Drive, Edgewood, N.Y. 11717-8329, TCG-MA 100H2 Fiber at OFS Fitel LLC 2000 Northeast Expressway, 30071 USA). The display shows the results of the calculations as a medically relevant value for determining the blood oxygenation of the patient and whether medical intervention via oxygen therapy is appropriate to correct low arterial blood oxygenation. See, e.g., FIG. 14. See also FIG. 16, which shows how a standard “Edwards” set up would be configured to work with embodiments of the invention.

The invention can take different forms and embodiments. Variations obvious to those skilled in the art will be included within the invention.

For example, the embodiment illustrated in FIG. 2 has the dimensions indicated. Double optic fiber 2 has an outer diameter approximately one-half the inside diameter of lumen 23 (the inside space of the cannula of catheter 22). This leaves a substantial free space inside the cannula. As can be appreciated from FIG. 2, a cross-sectional diameter of cable 2 of about ½ of the cross-sectional diameter of the lumen of the cannula of catheter 22 results in much more free cross-sectional area of lumen 23 relative to the cross-sectional area of cable 2. This also allows for easy threading of fiber optic cable 2 through the catheter (and removal therefrom), including when catheter 22 is pre-placed in operative position in patient 28. Thus, the quite small outer diameter (˜300 micrometers OD) can provide the independent function of gathering oximetry information from arterial/venous blood of the patient without a second invasive procedure. It essentially shares the single lumen interior space of a conventional arterial catheter. It can be withdrawn independently of the catheter. It does not require modification of the catheter. However, some variation of relative size is possible.

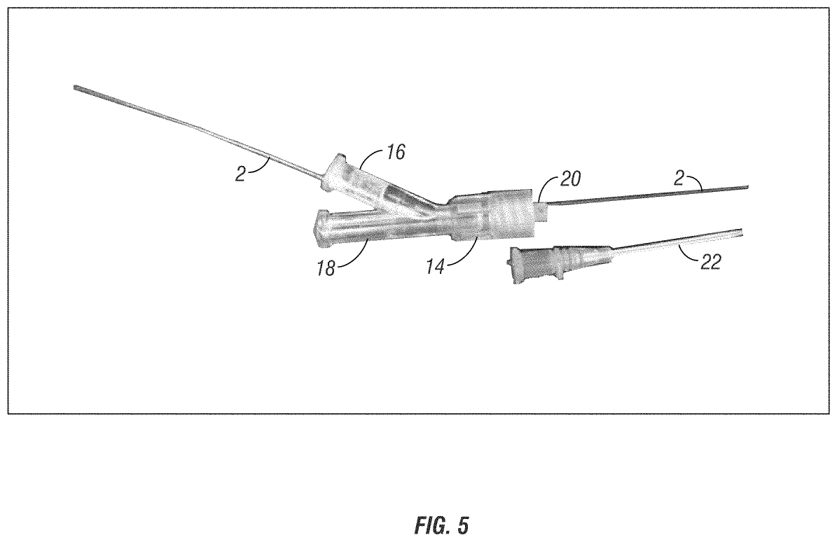

B. FIGS. 5-8

Further details and information about the embodiment of FIGS. 1-4 are as follows:

1. Details about Materials and Configuration:
Wrapping around optics: Polyester Shrink (known biocompatibility). Exemplar Manufacturer: Vention Medical.
Optic: 100 um core, 110 um cladding, and a 140 um polyimide buffer. Total OD of one optic=140 um.
Operating Temperature −65 to +300° C. Optics coated with “PYROCOAT” and appropriate cladding. Exemplar Manufacturer: OFS Optics (OFS part #F19113)
Y-Connector: Stock polycarbonate luer-lock. Exemplar Manufacturer: Vention Medical. A similar two-port connector (e.g. T-connector) could be used as well.

See also FIGS. 5-7, which are a photograph of a prototype (with catheter separated like FIG. 1) and additional cross-sections like FIG. 2.

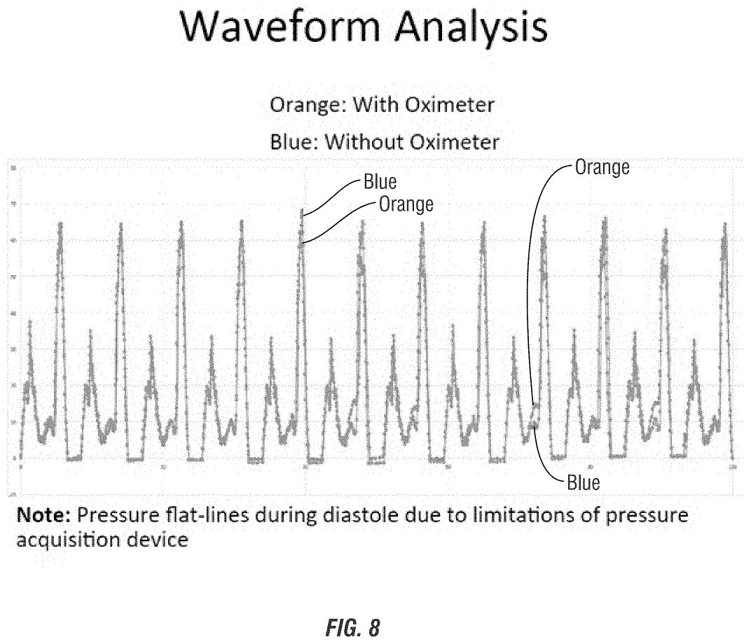

2. Testing Results (Graph Attached at FIG. 8):
On average there was between a 1 and 4 mmHg difference in systolic reading between measurements with oximeter in place or without oximeter in place (see FIG. 8).
A difference of 1-4 mm Hg is not clinically significant and can be seen with current arterial lines due to patient movement.
No statistically significant difference in pressure readings with or without oximeter in place.

3. Possible Options

Changes could include elongating the intravascular fiberoptic strands in order to be used with different types of arterial catheters such as neonatal umbilical arterial lines, the optics would need to be longer so that the tip of the optics still terminates at the tip of the catheter.

### II. Embodiment 2—ABG

This relates to using an apparatus at least similar to that of Embodiment 1 in measuring a variety of arterial blood gases (ABGs).

In one aspect, the method is for measuring arterial blood gas values (i.e. pH, PaCO2, PaO2, bicarbonate). It can use known technologies for determining the measurements from the returned optical signal from the apparatus.

For some background, see discussion of arterial blood gases below. See also, (Tintinalli's Emergency Medicine: Comprehensive Study Guide, Se Judith E. Tinfinalii, J. Stephan Stapczynski, O. John Ma, Donald M. Mealy, Garth D. Meckler, David M. Cline).

An arterial blood gas (ABG) test measures the acidity (pH) and the levels of oxygen and carbon dioxide in the blood from an artery. This test is used to check how well lungs are able to move oxygen into the blood and remove carbon dioxide from the blood.

As blood passes through the pulmonary capillary beds, oxygen moves into the blood while carbon dioxide moves out of the blood into the lungs. An ABG test uses blood drawn from an artery, where the oxygen and carbon dioxide levels can be measured before they enter body tissues. An ABG measures:
Partial pressure of oxygen (PaO2). This measures the pressure of oxygen dissolved in the blood and how well oxygen is able to move from the airspace of the lungs into the blood.
Partial pressure of carbon dioxide (PaCO2). This measures the pressure of carbon dioxide dissolved in the blood and how well carbon dioxide is able to move out of the body.
pH. The pH measures hydrogen ions (H+) in blood. The pH of blood is usually between 7.35 and 7.45. A pH of less than 7.0 is called acid and a pH greater than 7.0 is called basic (alkaline).
Bicarbonate (HCO3). Bicarbonate is a chemical (buffer) that keeps the pH of blood from becoming too acidic or too basic.
Oxygen content (O2CT) and oxygen saturation (O2Sat) values. O2 content measures the amount of oxygen in the blood. Oxygen saturation measures how much of the hemoglobin in the red blood cells is carrying oxygen (O2).

Blood for an ABG test is taken from an artery. Most other blood tests are done on a sample of blood taken from a vein, after the blood has already passed through the body's tissues where the oxygen is used up and carbon dioxide is produced.

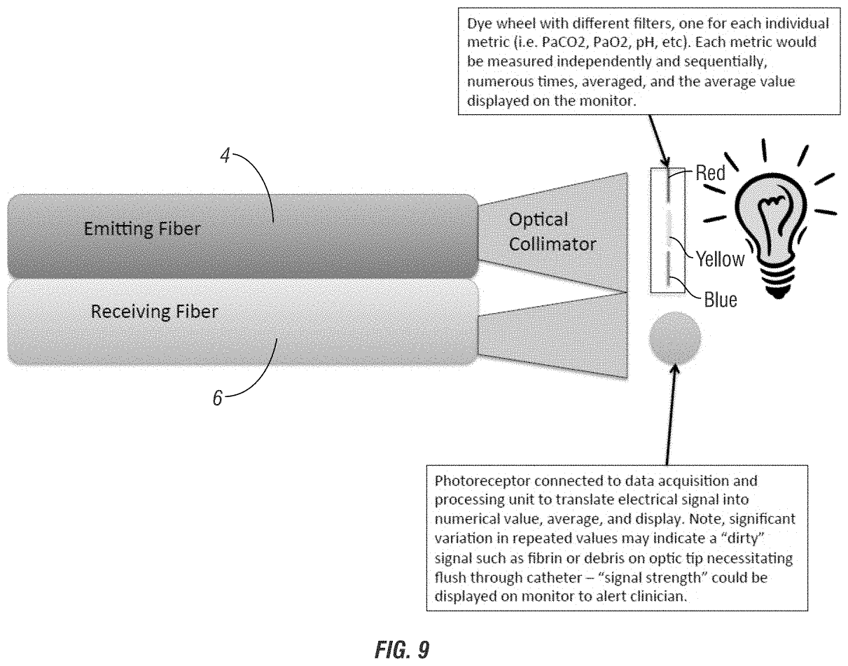

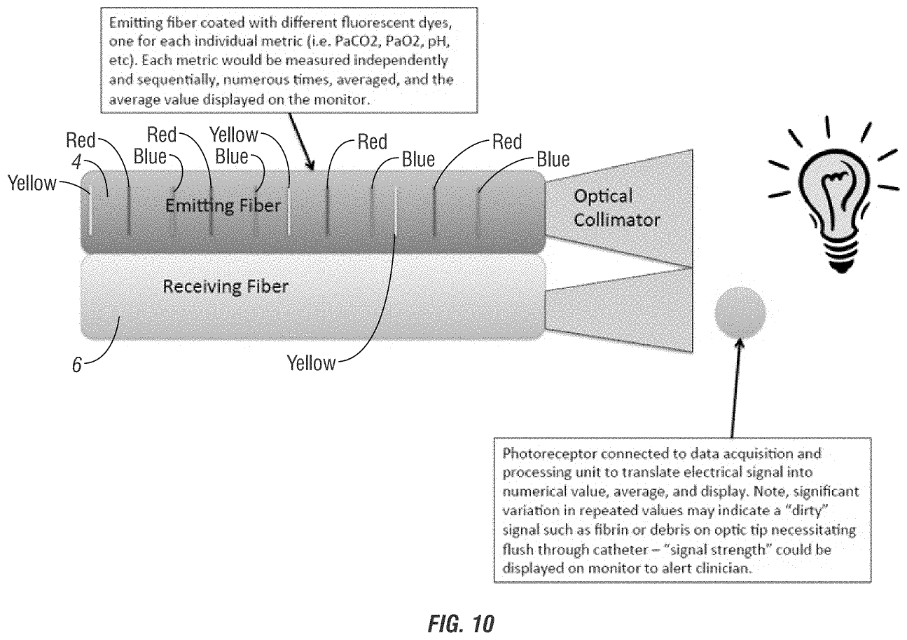

Utilizing optical fluorescence technology, each ABG (Arterial blood gas) metric is measured individually, stored and displayed as a set of metrics (e.g. pH, PaCO2, PaO2, HCO3). Each metric can be stored internally and then the set displayed together approximately every few minutes. Average measurements can be displayed to limit aberrancy in individual measurements.
See description of Optical Fluorescence Technology from Terumo at http://www.terumo-cvs.com/optimizing/2012OCT_OpticalFluorescenceTech.shtml incorporated by reference herein.
See also: U.S. Pat. No. 6,009,339 A (TERUMO) incorporated by reference herein.
See FIG. 9 of use of our probe in conjunction with optical fluorescence and color wheel.
See FIG. 10 of use of probe with emitting fiber coated in fluorescent dye which would be “excited” by illumination and then the excited electron reflectance would be received by the receiving fiber to the photodiode.
In essence, this relates to this probe could be used in conjunction with this existing platform in order to continually measure ABG values at the bedside without needing lab draws. This probe is another method by which to use basic technology of Embodiment 1. Note, use in a venous catheter to yield PvO2, PvCO2, venous pH is included.

### III. Embodiment 3—Hemoglobin

Utilizing Near-Infrared spectroscopy otherwise known as “rainbow” spectroscopy, multiple wavelengths of light from approximately 500-1,100 nm would be analyzed using this dual-fiber system, one sending fiber and one receiving fiber. Red and Infrared light is used to attain these wavelengths. Multiple wavelengths would be analyzed to gather both oxyhemoglobin (approximately 950 nm, infrared) and deoxyhemoglobin (approximately 650 nm red light) measurements. These measurements (amount of light reflected back to the photodiode receiving (afferent) strand) would then be calculated to derive a total hemoglobin value. Total hemoglobin is the sum of oxyhemoglobin and deoxyhemoglobin (HbT=HbO2+Hb).
See also: U.S. Pat. No. 7,613,489 B2 (HUTCHINSON TECHNOLOGY INC) and U.S. Pat. No. 6,144,444 A (MEDTRONIC) both incorporated by reference herein.

In essence, this claim is stating that this probe could be used in conjunction with this existing platform in order to continually measure hemoglobin/hematocrit values without needing lab draws. This probe is another method by which to use this existing technology advantageously.

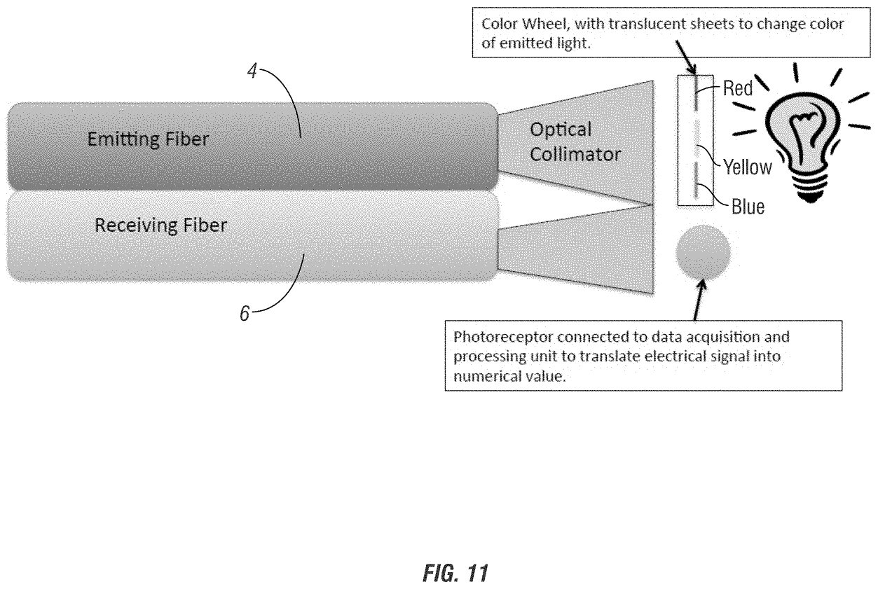

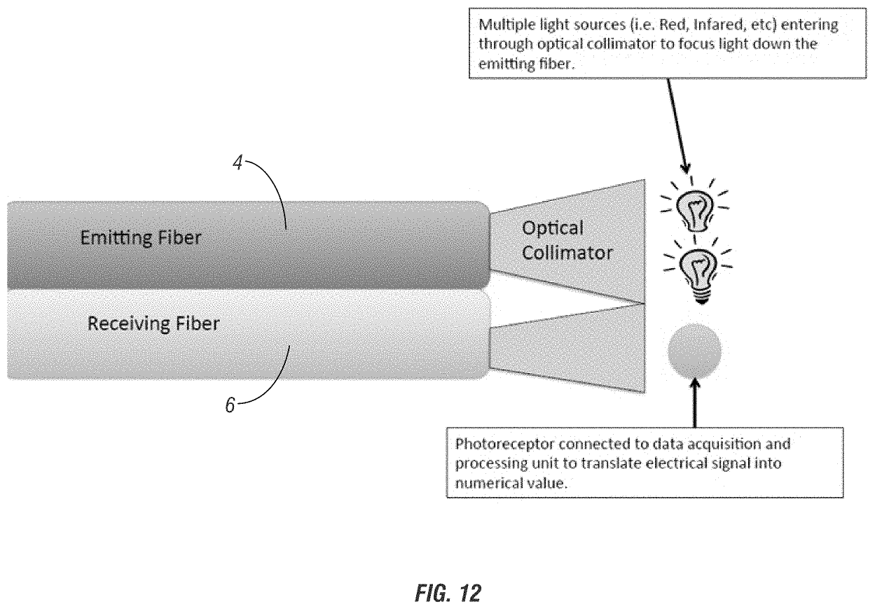

See, for example, FIGS. 11 and 12, which relate to alternative ways to introduce different light wavelengths into the emitting fiber of the dual fiber optic combination. FIG. 11 shows a color wheel of different sections that could be selectively moved between a starting light source and the entrance to the emitting fiber. FIG. 12 shows multiple light sources which could be selectively turned on (one at a time, or in any combination) to alter the light into the emitting fiber.

### IV. Embodiment 4—Solid Tissue

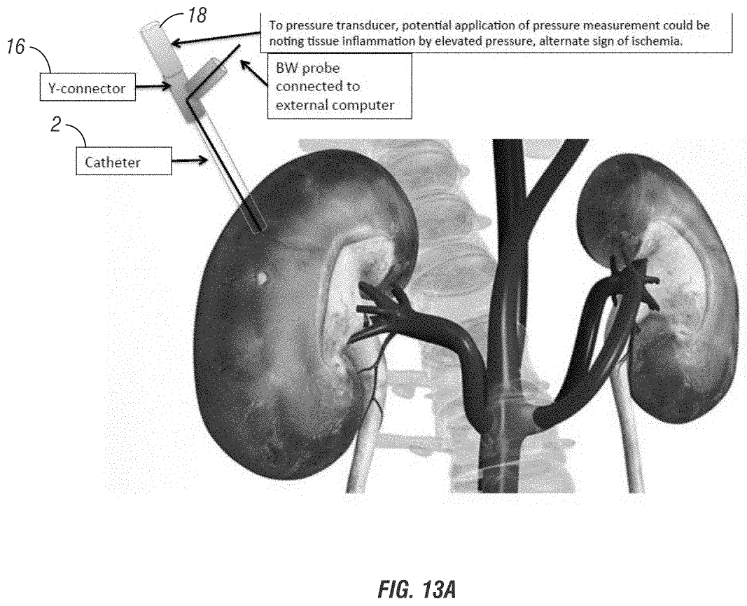

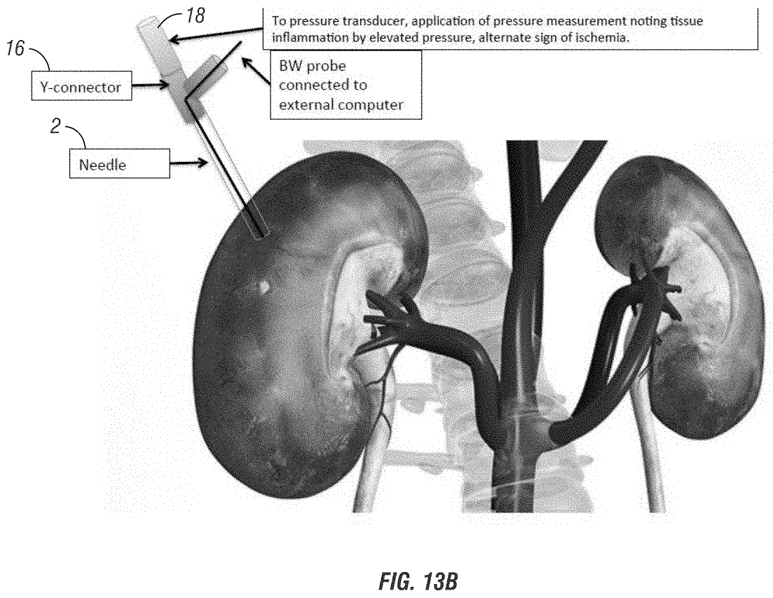

The dual-fiber probe would be inserted into a solid tissue structure such as the kidney (see “Solid tissue” diagrams of FIGS. 13A and B) through the inner lumen of a needle (FIG. 13B). The needle would be used to provide support to the optics so that they do not bend or kink, a firm catheter could be used (FIG. 13A). The fiberoptic tip would be at the tip of the needle/catheter, or extended slightly in front or behind the needle tip. The same principal of oximetry as described in the original claims would be utilized here for direct tissue oximetry. The pressure port of the Y-connector would be hooked up to a standard pressure transducer to note increase/decrease in pressure in the solid tissue indicating inflammation (increase) and possible necrosis.

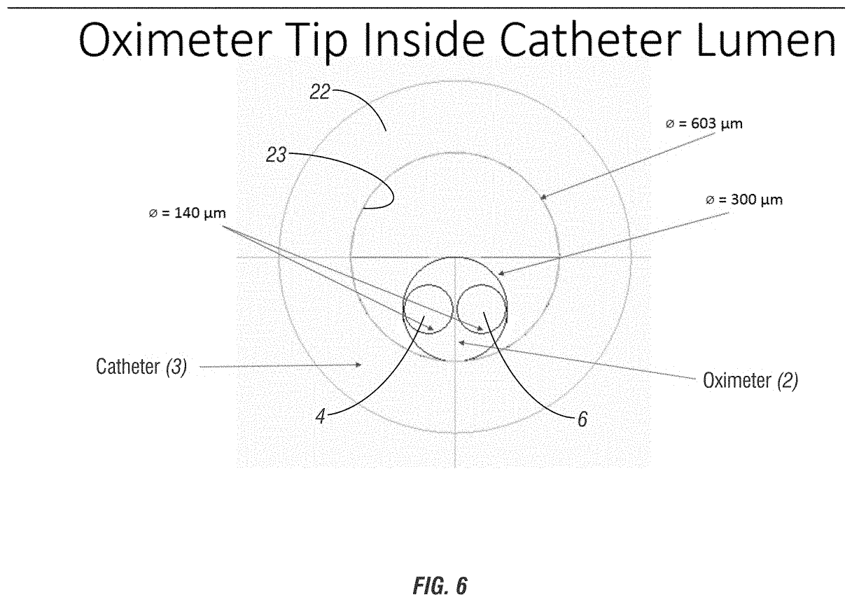

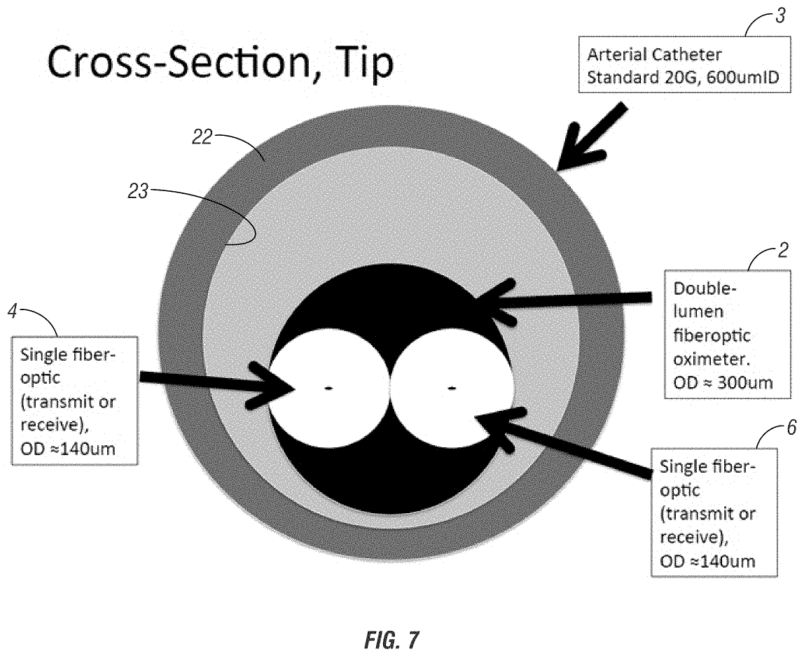

Additionally the Oximeter cross section tip diagrams (e.g. FIGS. 6 and 7) are included to show the relationship between the size of the dual-optic probe and the catheter, or needle (if used in solid structure). The optic outer diameter is approximately ½ the inner diameter of the catheter/needle. Note that bench testing confirms that there is no difference in pressure transduction with or without the oximeter probe in place; on the order of half the inner diameter of the catheter/needle should be reserved for the optics.

### V. Other Design Considerations

Below is additional discussion regarding possible structure, use, methodologies, and considerations for a designer or user of at least some of the embodiments. It is to be understood by the reader that the invention can take many forms and embodiments. This includes variations such as are obvious to those skilled in the art.

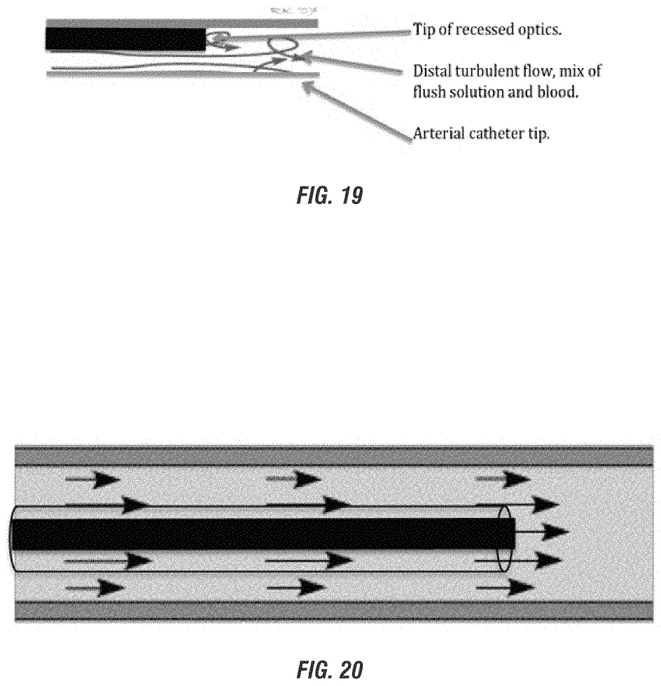

The designer may be faced with the following issues regarding some embodiments of the invention:
Optic tip location discrepancy
   Originally the optic tip was hypothesized to be best if slightly recessed from the tip of the arterial catheter. While it was claimed that this location provided the “best” results, the term “best” was not quantified.
Discrepancy in PaO2 and PaCO2 metrics
   Concern that metrics could have shown discrepancy due to contamination from heparinized saline on the optic tip.
Added complexity of passive compliance device
   Passive compliance device was reportedly added to allow fresh blood to be exposed to the optics; it was reported that the passive compliance device was adjusted to begin to flatten the dicrotic notch on the arterial tracing.
Use of three optics necessitates smaller optics be used.
   Proposed solutions to the above obstacles using this described probe:
Optic tip location discrepancy
   Recessing the optic tip slightly in the arterial catheter may create an unwanted turbulent flow at the optic tip. This unwanted turbulent flow is now a mixture of saline/heparin flush solution and fresh blood.
   This is the same concept as an intravascular plaque which causes turbulent flow just distal to the plaque.
   See FIG. 19 which schematically shows this possible effect.
   Proposed Solution: This probe tip resides flush with the tip of the arterial catheter. This will decrease the excess turbulent flow and the unwanted mixing of flush solution and blood.
      To decrease turbulent flow with a recessed tip, the flush solution rate would need to be decreased which would greatly increase risk for thrombus formation.
      Decreasing turbulent flow by placing the tip flush with the catheter may also decrease concern for any “whipping” of the probe tip in the catheter, thereby stabilizing the probe and the signal it is recording.
      See FIG. 20 which shows this as a possible solution.
         This figure (FIG. 20) shows the arterial catheter with the probe extending to its tip. There is no area of reduced flow as compared to the systemic arterial flow as there is with the recessed tip. Therefore any concern for turbulence is greatly reduced or eliminated.
Discrepancy in PaO2 and PaCO2 metrics
   As mentioned above, positioning of the probe tip flush with the catheter tip will decrease turbulent flow and mixing of flush solution and fresh blood. These variables are most likely the cause for aberrant value recordings.
   Proposed Solution:
      Proper positioning of fiber-optic probe tip as noted above.
      Displayed arterial blood gas (ABG) metrics could be calculated as averages of multiple collected values over a period of time. Note, the time period to collect a few values should be relatively short, 1-5 minutes. Individual values could be collected and stored locally, calculated into averages and displayed every certain time period. For example, 10 PaCO2 values could be collected, one every 30 seconds for 5 minutes and then 1 PaCO2 value is displayed every 5 minutes along with all other ABG values; one full ABG would be displayed every 5 minutes.
         Clinically, it would be acceptable to have more accurate values slightly less frequently than more aberrant values more frequently.
         Example: On patients in the ICU who do not have an arterial line who are relying on NIBP measurements, every 15 minute measurements are standard in practice. Therefore, it could be acceptable that an ABG only be displayed every few minutes as compared to every few seconds and still be clinically beneficial and relevant.
         Added complexity of Passive Compliance Device
         This component was added to provide oscillatory flow of fresh blood to the recessed optic tip.
         Proposed Solution: By keeping the optic tip flush with the tip of the catheter there is no need to provide oscillatory flow because there will always be fresh blood flowing over the optic tip. This eliminates the need for this added complexity.
Use of three optics necessitates smaller optics used.
   Proposed Solution: Utilizing the dual-optic probe provides with one sending and one receiving fiber, This allows larger optics for each to be used, which potentially leads to a stronger received signal and more reliable data.
      Use of a filter wheel as mentioned in the article.
      See: FIG. 9.

### Additional Proposals to Consider:

Continuous SaO2 and Hemoglobin measurement/trending in the ICU does have a profound utility. The peripheral intra-arterial probe could be used in conjunction with existing ABG and hemoglobin monitoring platform technology (for example, Terumo CDI 500 Blood Parameter Monitoring System). This technology on a miniaturized scale to provide real-time SaO2 and Hemoglobin measurement at the bedside. Providing this real-time metric would decrease lab draws (i.e. invasive line accessing and risk for infection, venipuncture, patient discomfort), healthcare-induced anemia, and healthcare spending on repeated lab tests.
Monitoring of only SaO2, pH and PaCO2 may be clinically sufficient. It may not be essential to measure PaO2 if a reliable SaO2 metric is measured. Based on the oxyhemoglobin-disassociation curve, with a known hemoglobin and SaO2, a PaO2 can be estimated. Essentially, a PaO2 over 70-80 mmHg may be irrelevant data as the hemoglobin are already fully loaded and dissolved oxygen does not provide much, if any, supplemental tissue oxygenation.
Minimally invasive tissue monitoring during cardiopulmonary bypass. Utilizing standard optical reflectance technology, as is used in current oximeter probes, the minimally invasive fiber-optic probe could be inserted into a solid tissue of concern, such as the kidneys during cardiopulmonary bypass to observe for inadequate renal perfusion. By placing the probe directly in the concerning tissue, current limitations of sensor spacing (i.e. distance between emitting light and receiving photodiode on current transcutaneous oxygenation probes), would be dramatically decreased or even eliminated. The probe could be inserted through a small sheath inserted over a needle into the tissue (See: FIGS. 13A and B).

In operation the probe can be inserted into an artery or vein or tissue. In one application, the probe is inserted and connected as described below. It is to be understood this is one example. Variations are possible.

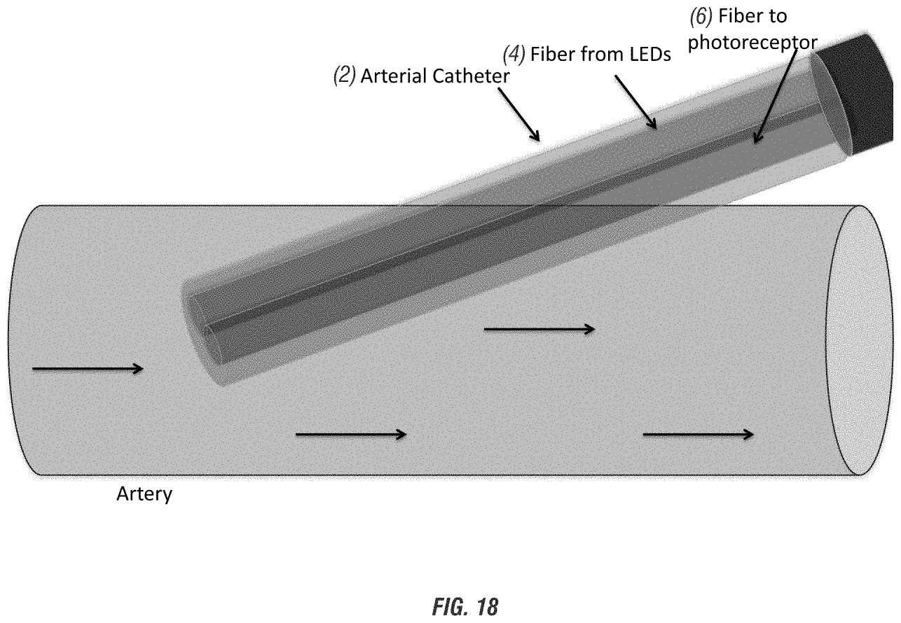

One orientation is as shown in FIG. 18 (e.g. an angle and with distal end pointed upstream of direction of blood flow). Additionally:
The two optical fibers are wrapped together, one connected to an LED, one to a photoreceptor. The strands or bundle are connected to a Y-connector (or other) which attaches to a hub of any arterial catheter. The combination can be added or removed to any arterial catheter. The same is true for venous catheters and applications.
In this example, the LED and photoreceptor are outside of the body.
The fiber optic tip is even with the catheter tip so that the fibers are exposed to fresh blood for best measurements. See also FIGS. 19 and 20.
   As will be appreciated by those skilled in the art, the invention can take many forms and embodiments. Variations such as those that are obvious to those skilled in the art will be included within the invention.

1-10. (canceled)

11. An oximeter device comprising:
a. a luer-lock arterial or venous catheter having a lumen between a first end and a second end;
b. a fiber optic cable containing a first fiber optic strand or bundle and a second fiber optic strand or bundle located within the lumen of the catheter and having a first end oriented with and extending just beyond, or at, said first end of said catheter and a second end passing through a three-port luer-lock connector, the fiber optic cable occupying a fraction of the lumen to allow the lumen other uses;
c. said three-port luer lock connector comprises a first channel, a second channel, and an end channel wherein the first channel connects to an IV line or arterial pressure transducing tube, the second channel has said fiber optic cable affixed to the interior walls, said second channel with the first end of the fiber optic cable extending through said end channel and said second end extending though said second channel, and wherein the end channel is connected to said second end of said catheter;
d. a light source connected to said first fiber optic strand or bundle oriented at the second end of said fiber optic strand or bundle cable;
e. a photo detector connected to said second fiber optic strand or bundle oriented at second end of said fiber optic cable in order to detect any light transmitted from said light source, through said first fiber optic strand or bundle and into said second fiber optic strand or bundle of said fiber optic cable;
f. an integrator connected to the light detector that receives a signal from the light detector and transforms it into an electronic signal; and
g. a display connected to the integrator that receives an electronic signal from it in order to display the amount of light transmitted as a medically relevant measure.

12. The oximeter of claim 11, wherein the other uses of the arterial/venous catheter include withdrawal of fluid, infusion of fluid, or another device threaded through the lumen of the arterial/venous catheter.

13. A method of invasively probing blood vessels or tissue comprising:
a. inserting a cannula into a blood vessel or tissue, the cannula having a single lumen with a lumen diameter and length;
b. threading a set of a plurality of encased parallel fiber optics along the lumen length into and through the single lumen to at or near its distal end, the set of plurality of encased fiber optics having an outer diameter which is a fraction of the lumen diameter;
c. operatively connecting the set of encased fiber optics to an external analysis system for processing reflectance captured from emitted light via the optical fibers and sharing the single lumen of the catheter cannula for optical interrogation and other catheter functions.

14. The method of claim 13 wherein the fraction is on the order of one half.

15. The method of claim 13 wherein the set of encased fiber optics is independently insertable and removable to and from the cannula and does not require modification of the cannula.

16. The method of claim 13 further comprising the cannula comprises:
a. an arterial or venous catheter or neonatal umbilical arterial line, or
b. a needle.

17. The method of claim 13 wherein the analysis system comprises:
a. one or more arterial blood gas analysis;
b. hemoglobin analysis; or
c. solid tissue analysis.

18. The method of claim 17 wherein the arterial blood gas analysis relates to at least one of PaCO2, PaO2, pH, PvCO2, PvO2 and further comprises:
a. one of:
i. a dye or color wheel with different filters at an entrance to an emitting optical fiber of the set of fiber optics;
ii. plural light sources of different color or characteristics at an entrance to an emitting optical fiber of the set of fiber optics; or
iii. different fluorescent dyes coated on an emitting fiber optic of the set of fiber optics, one for each type of arterial blood gas analysis, to alter the light source into or in the emitting fiber; and
b. a photoreceptor connected to data acquisition and processing unit to translate return light in a receiving fiber of the set of fiber optics for conversion into a numerical value, average, and/or display.

19. The method of claim 17 wherein hemoglobin analysis relates to utilizing near infrared spectroscopy with multiple wavelengths of light through the parallel fiber optics.

20. The method of claim 17 wherein the solid tissue analysis relates to use of the catheter and parallel fiber optics for optical interrogation of solid tissue.

21. The method of claim 13 wherein the cannula comprises a venous catheter and the encased fiber optics are for measuring SvO2, and further comprising:
a. inserting the venous catheter into a peripheral vein; and
b. using intravascular oximetry to measure venous oxyhemoglobin saturation (SvO2) which is placed in said venous catheter.

22. The method of claim 13 wherein the cannula comprises an arterial catheter and the encased fiber optics are for measuring SaO2, and further comprising:
a. inserting the arterial catheter into a peripheral artery with a distal end of the catheter facing the direction of blood flow;
b. using intravascular oximetry to measure arterial oxyhemoglobin saturation (SaO2) which is placed in said arterial catheter.

23. The method of claim 13, wherein
a. the fiber optics comprise a dual fiber optic strand/bundle threaded through the interior of the cannula of an arterial/venous catheter; and
b. the fiber optics occupy a fraction of the cross sectional inside diameter of the cannula of the arterial/venous catheter, no greater than one-half, to allow concurrent or other use of the catheter.
